(19) United States
(12) Patent Application Publication
(10) Pub. No.: US 2018/0111286 A1
(43) Pub. Date: Apr. 26, 2018

(54) Process of Improving the Dimensional Stability of Wood and Dimensional Stabile Wood Thereof

(71) Applicant: Kavita Sanghi, Pitampura (IN)

(72) Inventor: Kavita Sanghi, Pitampura (IN)

(21) Appl. No.: 15/822,328

(22) Filed: Nov. 27, 2017

(30) Foreign Application Priority Data
Feb. 4, 2007 (IN) 201611004022

(51) Int. Cl. B27K 3/15; B27K 3/08; B27K 3/50; B27K 5/00; B27K 5/02

(52) U.S. Cl. CPC B27K 3/15; B27K 3/08; B27K 3/50; B27K 2240/70; B27K 5/0055; B27K 5/02; B27K 5/001

(57) ABSTRACT

The present invention describes a process of improving the dimensional stability of wood and a dimensionally stable wood thereof. The said process is governed by contacting a composition comprising resin, catalyst and dye with a wood in a closed container followed by evacuating the air from the chamber and creating a vacuum. It is further treated with a composition comprising resin and dye followed by vacuum treatment during the contact step for at least 2 hours followed by a resting period and further treated in a temperature of approximately 100 degrees Celsius for about 1 hour and 30 minutes in order to obtain the desired wood.

### FIELD OF THE INVENTION

The present invention pertains to a process of treatment of wood.

More particularly, it relates to the process of stabilization of wood to increase its dimensional stability and decrease its hygroscopicity.

### BACKGROUND AND PRIOR ART OF THE INVENTION

Wood has been one of the most extensively used substances in the past few decades. It has been used for a wide variety of purposes ranging from making furniture and tools to being used as a fuel.

However, degradation of wood due to bacteria, fungi, etc. changes the properties of wood and makes it dry, and brittle. This has been a major concern that must be addressed. Accordingly, a large number of wood treatment processes have been developed to preserve the quality of wood and increase it's life and durability.

Wood is mainly composed of cellulose, hemicellulose and lignin composition of the cellulose and hemicellulose molecules exist A large number of hydroxyl groups, so that the internal water timber will change with the ambient temperature and humidity changes, and wood In all directions or internal moisture absorbent in the desorption process causes shrinkage of the wood swelling occurs, so that the timber with the timber Changes in moisture content and uneven expansion and contraction prone to warping, deformation, cracks and other defects, which restricts the range of application of wood.

Therefore, to improve the dimensional stability of the wood has an important significance for the efficient use of wood.

One of the wood treatment processes is wood stabilization, wherein wood is impregnated with solvent solutions of certain chemical compounds to make it water resistant as well as increase its density and durability. Such processes normally involve cold soaking or forcing solvents into wood under high pressure, and subsequently exposing it to high temperatures to produce stabilized wood.

Reference may be made from the Chinese Patent Publication No. CN102431067B which discloses a Pretreatment species increase pressure superheated steam timber dimensional stability, into the timber through the stack temperature, the wood pile Temperature, wood core values and the relationship between temperature control, complete pretreatment process.

Another reference may be made from CN105082282 which is related to a method of improving the dimensional stability of wood, characterized in that, the timber comprising soaking in petroleum wax, on wood Material injection-wax soaking wood absorb petroleum wax.

Another reference may be made from US Patent Application No. US20130171358, which discloses a wood treatment method and apparatus utilizing a treatment vessel having an integrated bundle hold-down system for securing a bundle of wood within the interior of the vessel during treatment. The bundle hold-down system can be operable to counteract large buoyant forces associated with submersing the bundle of wood in a liquid reagent and/or can be used to maintain the shape and/or position of the bundle during treatment, even if a liquid reagent is not present.

Further, reference may be made from PCT/US2015/042073 which discloses the use of acetic acid during wood treatment requires stainless steel, corrosion In addition, residual acid in the treated wood product requires the use of stainless steel and corrosion-resistant metal fastener hardware. Acetylated wood is also extremely prone to mold growth when exposed to a moist environment. As a result, acetylated wood requires a surface protective coating or colorants if used in an outdoor condition.

Furfurylation generally provides treated wood with an ASE of about 60% and a weight gain of about 30%. Furfurylation processes and furfulated wood release undesirable volatile organic compounds (VOC) during the curing process. In addition, furfurylation also results in increased brittleness of the wood. The characteristics, expense and complexity of these processes for enhancing the dimensional stability of wood limit the commercial usefulness of these processes.

However, some of the above methods Timber size can not fundamentally solve the problem of instability, the introduction of chemical modifiers of some chemical modification process Environment and people's health caused some harm, it is difficult to be accepted by consumers. Also, treating wood with chemical compounds changes the properties of wood itself.

Thus, an improved and efficient process of wood treatment and stabilization is required to create highly durable wood that is easy to machine and turned into various products.

### OBJECTS OF THE INVENTION

The main object of the present invention is to provide a process for improving the dimensional stability of wood.

It is another object of the present invention to provide a process for stabilization of wood that increases the dimensional stability, density and the durability of the wood.

It is yet another object of the present invention to provide a process for stabilization of wood that reduces the hygroscopicity of the wood.

It is further another object of the present invention to provide a process for stabilization of wood that produces stabilized wood that can be easily machined to be shaped into cylindrical and circular objects.

It is still another object of the present invention to provide an economical and efficient process for stabilization of wood.

It is still another object of the present invention to provide a dimensionally stable timber or wood.

### SUMMARY OF THE INVENTION

The present invention provides a process of improving the dimensional stability of wood by contacting a composition comprising resin, catalyst and dye with a wood in a closed container followed by the vacuum. It is further treated with composition comprising resin and dye followed by vacuum treatment during the contact step for at least 2 hours followed by resting period and further treated in temperature of approximately 100 degree Celsius in order to obtain the desired wood.

The present invention also provides a dimensionally stable wood with improved dimensional stability.

### BRIEF DESCRIPTION OF THE DRAWINGS

Embodiments of the present invention will hereinafter be described in conjunction with the following drawing FIGURE wherein:

FIG. 1 schematically illustrates a process of improving the dimensional stability of wood in accordance with an exemplary embodiment.

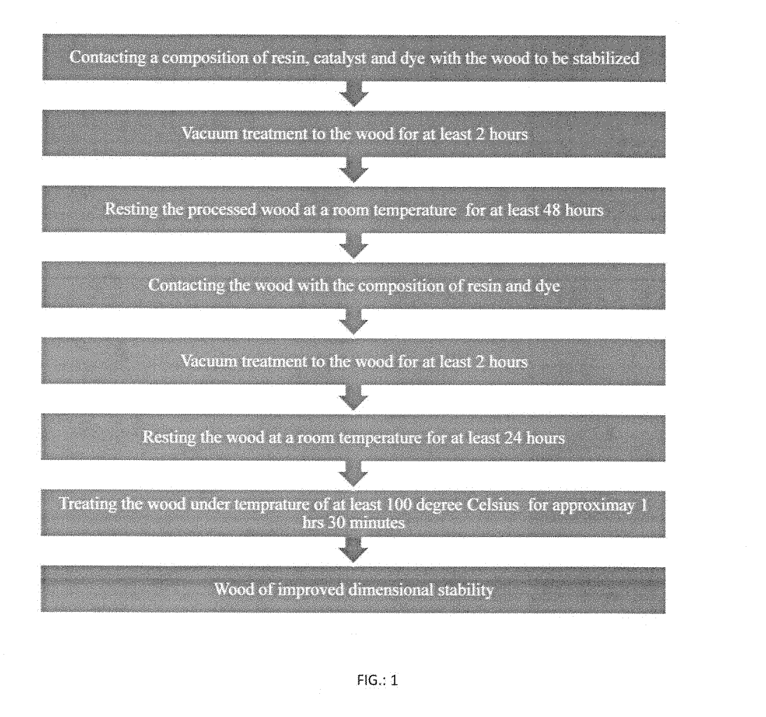

The above summary, as well as the following detailed description of certain embodiments of the present invention, will be better understood when read in conjunction with the appended drawings. For the purpose of illustrating the invention, certain embodiments are shown in the drawings. It should be understood, however, that the present invention is not limited to the arrangements and instrumentality shown in the attached drawings.

### DETAILED DESCRIPTION OF THE INVENTION

Accordingly, the present invention provides a process of improving the dimensional stability of wood, wherein the said process comprising the steps of:
a) contacting a composition comprising resin, catalyst and dye with a wood in a closed container;
b) applying vacuum treatment to the wood of step (a) during the contact step for at least 2 hours;
c) resting the wood of step (b) at a room temperature for at least 48 hours;
d) contacting the wood of step (c) with the composition comprising resin and dye followed h vacuum treatment during the contact step for at least 2 hours;
e) resting the wood of step (d) at a room temperature for at least 24 hours;
f) treating the said wood of step (e) with heat of at least 100 degree Celsius for approximately 1 hrs 30 minutes followed by gradually decreasing of heat for about 6 hours in order to obtain the desired wood of improved dimensional stability; wherein the said wood has a size at least 1.5 mm diameter greater than the diameter of the wood used in step (a).

In an embodiment of the present invention, the catalyst used to the Resin is in the range of 2.1 gm/lit to 2.3 gm/lit.

In yet another embodiment of the present invention, the resin preferably used is Resinol 90C.

In still another embodiment of the present invention, the dye used is selected from the group of oil solubles dyes.

In still another embodiment of the present invention, the dye used is selected as per the shade of the wood is desired.

In still another embodiment of the present invention, the heat is produced by the oven, electric tandoor, microwave oven etc.

The present invention also provides a dimensionally stable wood prepared by the above process.

The following describes example embodiments of the present invention and any alternatives or embodiments not specifically disclosed herein, but as may be obvious to persons skilled in the art, are encompassed by this disclosure, and fall within the scope of this invention.

The method of stabilization of wood, in accordance with example embodiments of the present invention is described hereafter. The wood to be stabilized in the form of a plank, slate, board or the like is placed inside the stabilizing chamber. The invention encompasses stabilizing any form of wood, in any shape that is capable of being placed inside the stabilizing chamber.

Subsequently, the stabilizing chamber is then filled with one or more stabilizing materials, such that it partially or wholly covers said wooden plank.

The invention encompasses using stabilizing materials such as one or more resins, wherein in a preferred embodiment, Resinol 90C is used in this step.

Next, a vacuum is created within the stabilizing chamber with the help of a vacuum pump attached to said chamber. Creation of vacuum increases the pressure inside the stabilizing chamber, which leads to release of air bubbles from the stabilizing material covered wooden plank. This results in better impregnation of the stabilizing material into the wooden plank. The pressure inside the stabilizing chamber is increased until the air bubbles stop.

Thereafter, the wooden plank is taken out of the stabilizing chamber and is wrapped in a material such as an Aluminum foil, which is then placed inside a preheated oven for about one hour and 30 minutes. The oven is typically preheated to a temperature within the range of 70-100 degree Celsius, wherein in a preferred embodiment, the oven is preheated to a temperature of 100 degree Celsius. The invention encompasses other temperature ranges that may be known or obvious to a person skilled in the art. The time for which the wooden plank be placed inside a preheated oven may depend upon several factors such as the type of wood, the size and shape of the plank, the temperature of the oven, the stabilizing material used in the previous step, etc. Thus, other time ranges, other than 30 minutes, is also encompassed by the invention.

The wood thus produced as a result of the process of stabilization in accordance with the embodiments of the invention, is harder, stronger and can be easily machined to give a desired shape. In one embodiment, the shaped wood is used for producing knitting needles, or the like.

### Advantages of the Invention

The main advantages of the present invention are:
1) Present invention provides with cost effective dimensionally stable wood.
2) Present invention provides with high quality dimensionally stable timber which can directly that can be easily machined to be shaped into cylindrical and circular objects.
3) Present invention provides wood with improved dimensionally stable properties.

I claim:

1. A process of improving the dimensional stability of wood, wherein the said process comprising the steps of
a) contacting a composition comprising resin, catalyst and dye with a wood in a closed container;
b) applying vacuum treatment to the wood of step (a) during the contact step for at least 2 hours;
c) resting the wood of step (b) at a room temperature for at least 48 hours;
d) contacting the wood of step (c) with the composition comprising resin and dye followed by vacuum treatment during the contact step for at least 2 hours;
e) resting the wood of step (d) at a room temperature for at least 24 hours;
f) treating the said wood of step (e) with heat of at least 100 degree Celsius for approximately 1 hrs 30 minutes followed by gradually decreasing of heat for about 6 hours in order to obtain the desired wood of improved dimensional stability; wherein the said wood has a size at least 1.5 mm diameter greater than the diameter of the wood used in step (a).

2. The process as claimed in claim 1, wherein the catalyst to the Resin is in the range of 2.1 gm/lit to 2.3 gm/lit.

3. The process as claimed in claim 1, wherein the said resin preferably used is Resinol 90C.

4. The process as claimed in claim 1, wherein the dye used is selected from the group of oil soluble dyes.

5. The process as claimed in claim 4, wherein the dye used is selected as per the shade of the wood is desired.

6. The process as claimed in claim 1, wherein the heat is produced by the oven, electric tandoor, microwave oven etc.

7. A dimensionally stable wood, characterized in that the process according to claims 1-8 prepared by any one of the claim.

8. The process as claimed in claim 2, wherein the said resin preferably used is Resinol 90C
